(19) United States
(12) Patent Application Publication
(10) Pub. No.: US 2009/0047288 A1
(43) Pub. Date: Feb. 19, 2009

(54) Glucan compositions and methods of enhancing CR3 dependent neutrophil-mediated cytotoxicity

(76) Inventor: Jun Yan, Louisville, KY (US)

Correspondence Address: Biopolymer Engineering, Inc.;dba Biothera, Inc., c/o Intellevate, P.O. Box 52050, Minneapolis, MN, 55402, US

(21) Appl. No.: 11/818,697

(22) Filed: Jun. 15, 2007

Related U.S. Application Data
(60) Provisional application No. 60/814,148, filed on Jun. 15, 2006.

(51) Int. Cl. A61K 31/716; A61K 39/395; A61P 35/00

(52) U.S. Cl. 424/141.1; 514/54

(57) ABSTRACT

A 25 kD β-glucan composition is described herein that effects the CR3-dependent priming of neutrophils and can promote neutrophil killing of iC3b-opsonized targets. Also described herein are methods of enhancing neutrophil cytotoxicity locally at the site of the tumor.

### GOVERNMENT SUPPORT

The invention was supported, in whole or in part, by a grant NIH RO1 CA86412 from the National Institutes of Health and DAMD17-02-01-0445 from the US Army Breast Cancer Research Program. The Government has certain rights in the invention.

### BACKGROUND OF THE INVENTION

This application claims the benefit of U.S. Ser. No. 60/814,148 entitled GLUCAN COMPOSITIONS AND METHODS OF ENHANCING CR3DEPENDENT NEUTROPHIL-MEDIATED CYTOTOXITY, filed Jun. 15, 2006.

β-glucan is a complex carbohydrate, generally derived from several sources, including yeast, bacteria, fungi and cereal grains. Each type of β-glucan has a unique structure in which glucose is linked together in different ways, resulting in different physical and chemical properties. For example, β (1-3) glucan derived from bacteria and algae is linear, making it useful as a food thickener. The frequency of side chains, known as the degree of substitution or branching frequency, regulated secondary structure and solubility. β-glucan derived from yeast is branched with β(1-3) and P(1-6) linkages with 1,3 linked glucose on the side chains, enhancing its ability to bind to and stimulate macrophages (also referred as 1,3; 1,6 glucan). β-glucan purified from baker's yeast (Saccharomyces cerevisiae) is a potent anti-infective β-glucan immunomodulator.

The cell wall of S. cerevisiae is mainly composed of β-glucans, which are responsible for its shape and mechanical strength. While best known for its use as a food grade organism, yeast is also used as a source of zymosan, a crude insoluble extract used to stimulate a non-specific immune response. Yeast-derived P (1,3;1,6) glucans stimulate the immune system, in part, by activating the innate anti-fungal immune mechanisms to fight a variety of targets. Glucans are structurally and functionally different depending on the source and isolation methods.

β-glucans possess a diverse range of activities. The ability of β-glucan to increase nonspecific immunity and resistance to infection is similar to that of endotoxin. Early studies on the effects of β glucan on the immune system focused on mice. Subsequent studies demonstrated that β-glucan has strong immunostimulating activity in a wide variety of other species, including earthworms, shrimp, fish, chicken, rats, rabbits, guinea pigs, sheep, pigs, cattle and humans. Based on these studies, β-glucan represents a type of immunostimulant that is active across the evolutionary spectrum, likely representing an evolutionarily innate immune response directed against fungal pathogens. However, despite extensive investigation, no consensus has been achieved on the source, size, and form of β-glucan that is actually used in at least some of the immunostimulatory functions.

### SUMMARY OF THE INVENTION

β-glucan, a polysaccharide produced by barley and fungi including yeast, in combination with monoclonal antibodies hold promise for cancer therapy. β-glucans are bound by complement receptor 3 (CR3) and, in concert with target-associated complement fragment iC3b, elicit phagocytosis and killing of yeast. β-glucans may also promote killing of mammalian tumor cells bearing iC3b (which would be engendered by administration of anti-tumor mAbs). Described herein are methods of administration of β-glucan compositions to tumor bearing mice in combination with an anti-tumor mAb. This composition almost completely stops tumor growth. This activity derives from a 25 kD fragment of β-glucan released by macrophage processing of the parent polysaccharide. Unlike the parent β-glucan, which does not bind neutrophil CR3, the 25 kD β-glucan binds to neutrophil CR3, induces CBRM1/5 neoepitope expression, and elicits CR3-dependent cytotoxicity. These events require phosphorylation of the tyrosine kinase, Syk, and consequent phosphatidylinositol 3-kinase (PI 3-kinase) activation, because β-glucan-mediated CR3-dependent cytotoxicity is drastically decreased by inhibition of these signaling molecules. Thus, β-glucan enhances tumor killing through a cascade of events including in vivo macrophage cleavage of the polysaccharide, dual CR3 ligation and CR3-Syk-PI3-kinase signaling.

### BRIEF DESCRIPTION OF THE DRAWINGS

FIGS. 1A-1C graphically depict the tumoricidal activity of immunotherapy with β-glucan PGG in combination with anti-tumor mAbs. FIGS. 1A-1B are graphs of tumor diameter versus number of days post tumor implantation. FIG. 1C is a graph of percent survival versus number of days post tumor implantation.

FIGS. 2A-2F are a series of photographs and graphs showing macrophages accumulate soluble yeast β-glucan and process it to prime neutrophil complement receptor 3 (CR3). FIG. 2A is a series of photographs showing cells from spleen or bone marrow that were stained with F4/80-PE or anti-Gr-1-PE. FIG. 2B is a series of graphs showing percent cells with surface bound fluorescent β-glucan on particular days. The upper graph represents cells taken from the spleen, and the lower graph represents cells taken from the bone marrow. FIG. 2C is a series of photographs taken by confocal microscopy and also a series of graphs that represent the analysis of those cells by flow cytometry. FIG. 2D is a series of photographs that show peritoneal neutrophils stained with anti-Gr-1-PE. FIG. 2E is a graph showing the percentage of fluorescent positive neutrophils depicted in 2D. FIG. 2F is a graph showing the percentage of cytoxicity of the positive neutrophils depicted in 2D.

FIGS. 3A-3D are a series of photographs and graphs showing an excreted 25 kD β-glucan binds to neutrophil CR3, priming CBRM1/5 neoepitope induction and cytotoxicity. FIG. 3A is a series of graphs showing peritoneal neutrophils from wildtype (WT) and CR3^{-/-} mice that were stained with DTAF-labeled β-glucan PGG (upper panel) or with DTAF-labeled 25 kD active moiety released from macrophage culture (lower panel). FIG. 3B is a series of photographs showing human CR3 transfected CHO cells stained with anti-CR3-PE and DTAF-labeled parent β-glucan PGG or 25 kD β-glucan. FIG. 3C is a graph showing the percent of Gr-1⁺ cells with bound hexose-DTAF versus concentrations of hexose. FIG. 3D is a graph showing percentage of neutrophils with bound hexose-DTAF versus concentrations of Hexose.

FIGS. 4A-4C are a series of graphs showing that CR3 dual ligation leads to enhanced Syk phosphorylation, augmented PI 3-kinase activity and cytotoxicity. FIG. 4A shows proteins that were transferred onto nitrocellulose membranes and blotted with anti-Syk antibody or anti-phospho-Syk antibody. FIG. 4B is a series of graphs showing peripheral blood neutrophils that were stimulated with 25 kD β-glucan only, antibody only, or both and then assessed by flow cytometry. FIG. 4C is a graph showing the level of PI3P3 in cells stimulated with glucan only, antibody only, or both.

### DETAILED DESCRIPTION OF THE INVENTION

A description of preferred embodiments of the invention follows.

A 25 kD β-glucan composition is described herein that effects the CR3-dependent priming of neutrophils and can promote neutrophil killing of iC3b-opsonized targets. As shown herein, the 25 kD glucan and not the parent glucan enhances the cytotoxicity of neutrophils against iC3b-opsonized tumor cells. Also described herein are methods of enhancing neutrophil cytotoxicity locally at the site of the tumor. In certain embodiments, the glucan is used in combination with complement activating anti-tumor monoclonal antibodies, for example, rituximab and trastuzumab. Advantages include efficient, local delivery at the tumor site, direct delivery without any prior processing, development of sustained release composition, the number of administrations may be cut down to a weekly rather than daily basis and the glucan may be effective in immunologically-impaired individuals that are unable to efficiently metabolize the parent polysaccharide.

β-glucan, a well-known biological response modifier (BRM), stimulates hematopoiesis (blood cell formation) in an analogous manner as granulocyte monocyte-colony stimulating factor (GM-CSF). Described herein are methods and compositions of 25 kD β-glucan compositions in combination with antitumor monoclonal antibodies for enhanced tumor killing.

Antitumor monoclonal antibodies bind to tumors and tumor cells and activate complement, coating tumors with iC3B. Intraveneously administered yeast β1,3;1,6-glucan functions as an adjuvant for antitumor mAb by priming the inactivated C3b(iC3bZ) receptors (CR3; CD11b/CD18) of circulating granulocytes, enabling CR3 to trigger cytotoxicity of iC3b coated tumors. Recent data indicated that orally administered yeast β1,3;1,6-glucan potentiated the activity of antitumor mAb, leading to enhanced tumor regression and survival. (Hong, F. et al., J. of Immunology; 173:797-806 (2004)). A requirement for iC3b on tumors and Cr3 on granulocytes was confirmed in C3- or CR3-deficient mice models.

Parent β-glucan, as described herein, is a soluble yeast β-glucan comprised of a β-D-(1-3)-linked glucopyranosyl backbone with β-D-(1-6)-linked β(1,3) side chains. The length of the side chains is between about 2 and 5 glucose residues in length. Suitable examples of soluble forms of parent β-glucan are described in U.S. Pat. Nos. 7,022,685; 6,369,216; 6,117,850; 6,046,323; 5,817,643; 5,849,720; 5,811,542; 5,783,569; 5,663,324; 5,633,369; 5,622,940; 5,622,939; 5,532,223; 5,488,040 and 5,322,841, which are assigned to Biopolymer Engineering, Inc. Additional methods of preparing compositions of parent β-glucan are described below.

The compositions described herein comprise a 25 kD β-glucan comprised of a β-D-(1-3)-linked glucopyranosyl backbone with β-D-(1-6)-linked β(1,3) side chains. The 25 kD β-glucan is a modified cleavage or degradation product produced by macrophage processing of parent β-glucan or by alternative in vitro processes utilizing various forms of starting β-glucan materials. The molecular weight of the 25 kD β-glucan is approximate and based on comparison to dextran standards eluted from HPLC. Depending on the method and standard used to determine molecular weight, the molecular weight of the β-glucan fragment may vary.

Glucan Receptor Binding with CR3

The iC3b-receptor CR3 (also known as Mac-1, CD11b/CD18, or α_{M}β₂-integrin) was shown to have a β-glucan-binding lectin site that functioned in the phagocytosis of yeast cell walls by neutrophils, monocytes, and macrophages (Ross, G. D., et al., Complement Inflamm. 4:61-74 (1987) and Xia, Y., V. et al., J. Immunol. 162:2281-2290 (1999)). Mac-1/CR3 functions as both an adhesion molecule mediating the diapedesis of leukocytes across the endothelium and a receptor for the iC3b fragment of complement responsible for phagocytic/degranulation responses to microorganisms. Mac-1/CR3 has many functional characteristics shared with other integrins, including bidirectional signaling via conformational changes that originate in either the cytoplasmic domain or extracellular region. Another key to its functions is its ability to form membrane complexes with glycosylphosphatidylinositol (GPI)-anchored receptors such as Fc gammaRIIIB (CD16b) or uPAR (CD87), providing a transmembrane signaling mechanism for these outer membrane bound receptors that allows them to mediate cytoskeleton-dependent adhesion or phagocytosis and degranulation. Many functions appear to depend upon a membrane-proximal lectin site responsible for recognition of either microbial surface polysaccharides or GPI-linked signaling partners. Because of the importance of Mac-1/CR3 in promoting neutrophil inflammatory responses, therapeutic strategies to antagonize its functions have shown promise in treating both autoimmune diseases and ischemia/reperfusion injury. Conversely, soluble β-glucan polysaccharides that bind to its lectin site prime the Mac-1/CR3 of circulating phagocytes and natural killer (NK) cells, permitting cytotoxic degranulation in response to iC3b-opsonized tumor cells that otherwise escape from this mechanism of cell-mediated cytotoxicity. CR3 binds soluble fungal β-glucan with high affinity (5×10⁻⁸ M) and this primes the receptor of phagocytes or NK cells for cytotoxic degranulation in response to iC3b-coated tumor cells. The tumoricidal response promoted by soluble β-glucan in mice was shown to be absent in mice deficient in either serum C3 (complement 3) or leukocyte CR3, highlighting the requirement for iC3b on tumors and CR3 on leukocytes in the tumoricidal function of β-glucans (Vetvicka, V., et al., J. Clin. Invest. 98:50-61 (1996) and Yan, J., V. et al., J. Immunol. 163:3045-3052 (1999)).

CR3 plays a very important role in the antitumor activity of β-glucan. The role of CR3 in mediating the response to glucan was shown by research into the mechanisms of neutrophil phagocytosis of iC3b-opsonized yeast. When complement C3b has attached itself to a surface, it may be clipped by a serum protein to produce a smaller fragment, iC3b. While iC3b has been “inactivated” and cannot function to form a membrane attack complex, it remains attached to the surface where it serves to attract neutrophils and macrophages which can phagocytize or otherwise destroy the marked (“opsonized”) cell. On the surface of neutrophils and macrophages are complement receptors (CR3) that bind to iC3b.

Stimulation of CR3-dependent phagocytosis or degranulation requires the simultaneous ligation of two distinct sites within CR3; one specific for iC3b and a second site specific for glucan. Parent glucan that is transformed to the size of 25 kD and brought to the tumor site binds to the lectin site of CR3 to activate immune cells bearing the receptor to trigger degranulation and or phagocytosis of the foreign material.

### Preparation of Parent and Starting Forms of β-Glucan

The glucan described herein can be made by various methods known to one skilled in the art. For example, the preparation of neutral soluble glucan (NSG) is described in U.S. Pat. No. 5,322,841, the disclosure of which is incorporated herein by reference. Briefly, this method involves treating whole glucan particles with a series of acid and alkaline treatments to produce soluble glucan that forms a clear solution at a neutral pH. The whole glucan particles utilized in this present invention can be in the form of a dried powder, prepared by the process described above. For the purpose of this present invention it is not necessary to conduct the final organic extraction and wash steps.

In the present process, whole glucan particles are suspended in an acid solution under conditions sufficient to dissolve the acid-soluble glucan portion. For most glucans, an acid solution having a pH of from about 1 to about 5 and a temperature of from about 20° to about 100° C. is sufficient. Typically, the acid used is an organic acid capable of dissolving the acid-soluble glucan portion. Acetic acid, at concentrations of from about 0.1 to about 5M or formic acid at concentrations of from about 50% to 98% (w/v) are useful for this purpose. Additionally, sulphuric acid can be utilized. The treatment is usually carried out at about 90° C. The treatment time may vary from about 1 hour to about 20 hours depending on the acid concentration, temperature and source of whole glucan particles. For example, modified glucans having more β(1-6) branching than naturally-occurring, or wild-type glucans, require more stringent conditions, i.e., longer exposure times and higher temperatures. This acid-treatment step can be repeated under similar or variable conditions. In one embodiment of the present method, modified whole glucan particles from the strain, S. cerevisiae R4, which have a higher level of β(1-6) branching than naturally-occurring glucans, are used, and treatment is carried out twice: first with 0.5M acetic acid at 90° C. for 3 hours and second with 0.5M acetic acid at 90° C. for 20 hours.

The acid-insoluble glucan particles are then separated from the solution by an appropriate separation technique, for example, by centrifugation or filtration. The pH of the resulting slurry is adjusted with an alkaline compound such as sodium hydroxide, to a pH of about 7 to about 14. The slurry is then resuspended in hot alkali having a concentration and temperature sufficient to solubilize the glucan polymers. Alkaline compounds which can be used in this step include alkali-metal or alkali-earth metal hydroxides, such as sodium hydroxide or potassium hydroxide, having a concentration of from about 0.1 to about 10N. This step can be conducted at a temperature of from about 4° C. to about 121° C., typically from about 20° C. to about 100° C. In one embodiment of the process, the conditions utilized are a 1N solution of sodium hydroxide at a temperature of about 80°-100° C. and a contact time of approximately 1-2 hours. The resulting mixture contains solubilized glucan molecules and particulate glucan residue and generally has a dark brown color due to oxidation of contaminating proteins and sugars. The particulate residue is removed from the mixture by an appropriate separation technique, e.g., centrifugation and/or filtration.

The resulting solution contains soluble glucan molecules. This solution can, optionally, be concentrated to effect a 5 to 10 fold concentration of the retentate soluble glucan fraction to obtain a soluble glucan concentration in the range of about 1 to 5 mg/ml. This step can be carried out by an appropriate concentration technique, for example, by ultrafiltration, utilizing membranes with nominal molecular weight levels.

The concentrated fraction obtained after this step is enriched in the soluble, biologically active glucan, also referred to as β-glucan PGG. To obtain a pharmacologically acceptable solution, the glucan concentrate is further purified, for example, by diafiltration. In one embodiment, diafiltration is carried out using approximately 10 volumes of alkali in the range of about 0.2 to 0.4N. A suitable concentration of the soluble glucan after this step is from about 2 to about 5 mg/ml. The pH of the solution is adjusted in the range of about 7-9 with an acid, such as hydrochloric acid. Traces of proteinaceous material which may be present can be removed by contacting the resulting solution with a positively charged medium such as DEAE-cellulose, QAE-cellulose or Q-Sepharose. Proteinaceous material is detrimental to the quality of the glucan product, may produce a discoloration of the solution and aids in the formation of gel networks, thus limiting the solubility of the neutral glucan polymers. A clear solution is obtained after this step.

The highly purified, clear glucan solution can be further purified, for example, by diafiltration, using a pharmaceutically acceptable medium (e.g., sterile water for injection, phosphate-buffered saline (PBS), isotonic saline, dextrose) suitable for parenteral administration. The final concentration of the glucan solution is adjusted in the range of about 0.5 to 5 mg/ml. In accordance with pharmaceutical manufacturing standards for parenteral products, the solution can be terminally sterilized by filtration through a 0.22 μm filter. The soluble glucan preparation obtained by this process is sterile, non-antigenic, and essentially pyrogen-free, and can be stored at room temperature for extended periods of time without degradation.

In alternative methods for preparing particulate and soluble β-glucan, a yeast culture is grown, typically, in a shake flask or fermenter. In one embodiment of bulk production, a culture of yeast is started and expanded stepwise through a shake flask culture into a 250-L scale production fermenter. The yeast are grown in a glucose-ammonium sulfate medium enriched with vitamins, such as folic acid, inositol, nicotinic acid, pantothenic acid (calcium and sodium salt), pyridoxine HCl and thymine HCl and trace metals from compounds such as ferric chloride, hexahydrate; zinc chloride; calcium chloride, dihydrate; molybdic acid; cupric sulfate, pentahydrate and boric acid. An antifoaming agent such as Antifoam 204 may also be added at a concentration of about 0.02%.

The production culture is maintained under glucose limitation in a fed batch mode. During seed fermentation, samples are taken periodically to measure the optical density of the culture before inoculating the production fermenter. During production fermentation, samples are also taken periodically to measure the optical density of the culture. At the end of fermentation, samples are taken to measure the optical density, the dry weight, and the microbial purity.

If desired, fermentation may be terminated by raising the pH of the culture to at least 11.5 or by centrifuging the culture to separate the cells from the growth medium. In addition, depending on the size and form of purified β-glucan that is desired, steps to disrupt or fragment the yeast cells may be carried out. Any known chemical, enzymatic or mechanical methods, or any combination thereof may be used to carry out disruption or fragmentation of the yeast cells.

The yeast cells containing the β-glucan are harvested. When producing bulk β-glucan, yeast cells are typically harvested using continuous-flow centrifugation.

Yeast cells are extracted utilizing one or more of an alkaline solution, a surfactant, or a combination thereof. A suitable alkaline solution is, for example, 0.1 M-5 M NaOH. Suitable surfactants include, for example, octylthioglucoside, Lubrol PX, Triton X-100, sodium lauryl sulfate (SDS), Nonidet P-40, Tween 20 and the like. Ionic (anionic, cationic, amphoteric) surfactants (e.g., alkyl sulfonates, benzalkonium chlorides, and the like) and nonionic surfactants (e.g., polyoxyethylene hydrogenated castor oils, polyoxyethylene sorbitol fatty acid esters, polyoxyethylene sorbitan fatty acid esters, polyoxyethylene glycerol fatty acid esters, polyethylene glycol fatty acid esters, polyoxyethylene alkyl phenyl ethers, and the like) may also be used. The concentration of surfactant will vary and depend, in part, on which surfactant is used. Yeast cell material may be extracted one or more times.

Extractions are usually carried out at temperatures between about 70° C. and about 90° C. Depending on the temperature, the reagents used and their concentrations, the duration of each extraction is between about 30 minutes and about 3 hours.

After each extraction, the solid phase containing the β-glucan is collected using centrifugation or continuous-flow centrifugation and resuspended for the subsequent step. The solubilized contaminants are removed in the liquid phase during the centrifugations, while the α-glucan remains in the insoluble cell wall material.

In one embodiment, four extractions are carried out. In the first extraction, harvested yeast cells are mixed with 1.0 M NaOH and heated to 90° C. for approximately 60 minutes. The second extraction is an alkaline/surfactant extraction whereby the insoluble material is resuspended in 0.1 M NaOH and about 0.5% to 0.6% Triton X-100 and heated to 90° C. for approximately 120 minutes. The third extraction is similar to the second extraction except that the concentration of Triton X-100 is about 0.05%, and the duration is shortened to about 60 minutes. In the fourth extraction, the insoluble material is resuspended in about 0.05% Triton-X 100 and heated to 75° C. for approximately 60 minutes.

The alkaline and/or surfactant extractions solubilize and remove some of the extraneous yeast cell materials. The alkaline solution hydrolyzes proteins, nucleic acids, mannans, and lipids. Surfactant enhances the removal of lipids, which provides an additional advantage yielding an improved β-glucan product.

The next step in the purification process is an acidic extraction shown, which removes glycogen. One or more acidic extractions are accomplished by adjusting the pH of the alkaline/surfactant extracted material to between about 5 and 9 and mixing the material in about 0.05 M to about 1.0 M acetic acid at a temperature between about 70° C. and 100° C. for approximately 30 minutes to about 12 hours.

In one embodiment, the insoluble material remaining after centrifugation of the alkaline/surfactant extraction is resuspended in water, and the pH of the solution is adjusted to about 7 with concentrated HCl. The material is mixed with enough glacial acetic acid to make a 0.1 M acetic acid solution, which is heated to 90° C. for approximately 5 hours.

Next, the insoluble material is washed. In a typical wash step, the material is mixed in purified water at about room temperature for a minimum of about 20 minutes. The water wash is carried out two times. The purified β-glucan product is then collected. Again, collection is typically carried out by centrifugation or continuous-flow centrifugation.

At this point, a purified, particulate β-glucan product is formed. The product may be in the form of whole glucan particles or any portion thereof, depending on the starting material. In addition, larger sized particles may be broken down into smaller particles. The range of product sizes includes β-glucan particles that have substantially retained in vivo morphology (whole glucan particles) down to submicron-size particles.

As is well known in the art, particulate β-glucan is useful in many food, supplement and pharmaceutical applications. Alternatively, particulate β-glucan can be processed further to form aqueous, soluble β-glucan.

Particuate β-glucan starting material may range in size from whole glucan particles down to submicron-sized particles. The particulate β-glucan undergoes an acidic treatment under pressure and elevated temperature to produce soluble β-glucan. Pelleted, particulate β-glucan is resuspended and mixed in a sealable reaction vessel in a buffer solution and brought to pH 3.6. Buffer reagents are added such that every liter, total volume, of the final suspension mixture contains about 0.61 g sodium acetate, 5.24 ml glacial acetic acid and 430 g pelleted, particulate β-glucan. The vessel is purged with nitrogen to remove oxygen and increase the pressure within the reaction vessel.

In a particular embodiment, the pressure inside the vessel is brought to 35 PSI, and the suspension is heated to about 135° C. for between about 4.5 and 5.5 hours. It was found that under these conditions the β-glucan will solubilize. As the temperature decreases from 135° C., the amount of solubilization also decreases.

It should be noted that this temperature and pressure are required in the embodiment just described. Optimization of temperatures and pressures may be required if any of the reaction conditions and/or reagents are altered.

The increased pressure and temperature imparts advantages over prior art processes for solubilizing β-glucan by virtually eliminating the use of hazardous chemicals from the process. Hazardous chemicals that have previously been used include, for example, flammable VOCs such as ether and ethanol, very strong acids such as formic acid and sulphuric acid and caustic solutions of very high pH. The present process is not only safer, but, by reducing the number of different chemicals used and the number of steps involved, is more economical.

The exact duration of heat treatment is typically determined experimentally by sampling reactor contents and performing gel permeation chromatography (GPC) analyses. The objective is to maximize the yield of soluble material that meets specifications for high resolution-GPC(HR-GPC) profile and impurity levels, which are discussed below. Once the β-glucan is solubilized, the mixture is cooled to stop the reaction.

The crude, solubilized β-glucan may be washed and utilized in some applications at this point, however, for pharmaceutical applications further purification is performed. Any combination of one or more of the following steps may be used to purify the soluble β-glucan. Other means known in the art may also be used if desired. First, the soluble β-glucan is clarified. Suitable clarification means include, for example, centrifugation or continuous-flow centrifugation.

Next, the soluble β-glucan may be filtered. In one embodiment, the material is filtered, for example, through a depth filter followed by a 0.2 μm filter.

Chromatography may be used for further purification. The soluble β-glucan may be conditioned at some point during previous steps in preparation for chromatography. For example, if a chromatographic step includes hydrophobic interaction chromatography (HIC), the soluble β-glucan can be conditioned to the appropriate conductivity and pH with a solution of ammonium sulphate and sodium acetate. A suitable solution is 3.0 M ammonium sulfate, 0.1 M sodium acetate, which is used to adjust the pH to 5.5.

In one example of HIC, a column is packed with Tosah Toyopearl Butyl 650M resin (or equivalent). The column is packed and qualified according to the manufacturer's recommendations.

Prior to loading, the column equilibration flow-through is sampled for pH, conductivity and endotoxin analyses. The soluble β-glucan, conditioned in the higher concentration ammonium sulphate solution, is loaded and then washed. The nature of the soluble β-glucan is such that a majority of the product will bind to the HIC column. Low molecular weight products as well as some high molecular weight products are washed through. Soluble β-glucan remaining on the column is eluted with a buffer such as 0.2 M ammonium sulfate, 0.1 M sodium acetate, pH 5.5. Multiple cycles may be necessary to ensure that the hexose load does not exceed the capacity of the resin. Fractions are collected and analyzed for the soluble β-glucan product.

Another chromatographic step that may be utilized is gel permeation chromatography (GPC). In one example of GPC, a Tosah Toyopearl HW55F resin, or equivalent is utilized and packed and qualified as recommended by the manufacturer. The column is equilibrated and eluted using citrate-buffered saline (0.14 M sodium chloride, 0.011 M sodium citrate, pH 6.3). Prior to loading, column wash samples are taken for pH, conductivity and endotoxin analyses. Again, multiple chromatography cycles may be needed to ensure that the load does not exceed the capacity of the column.

The eluate is collected in fractions, and various combinations of samples from the fractions are analyzed to determine the combination with the optimum profile. For example, sample combinations may be analyzed by HR-GPC to yield the combination having an optimized HR-GPC profile. In one optimized profile, the amount of high molecular weight (HMW) impurity, that is soluble β-glucans over 380,000 Da, is less than or equal to 10%. The amount of low molecular weight (LMW) impurity, under 25,000 Da, is less than or equal to 17%. The selected combination of fractions is subsequently pooled.

At this point, the soluble β-glucan is purified and ready for use. Further filtration may be performed in order to sterilize the product. If desired, the hexose concentration of the product can be adjusted to about 1.0±0.15 mg/ml with sterile citrate-buffered saline.

### Preparation of the 25 kD β-Glucan

The 25 kD β-glucan is the product of macrophage processing of a parent β-glucan. To prepare the 25 kD β-glucan, macrophages are maintained in a bioreactor flask in macrophage growth serum-free medium, such as SFM medium, Invitrogen, Grand Island, N.Y. Labeled parent β-glucan is added to the culture. After about three weeks of cell culture, the cell-free fluid from the lower chamber of the bioreactor flask containing soluble fragments of β-glucan is collected. The β-glucan fragments are separated by, for example, high-performance liquid chromatography (HPLC) (Waters 1525, Waters Corp., Milford, Mass.) utilizing a monophasic gradient and separated on a Sephacryl S-200 (GE Healthcare, formerly Amersham Biosciences, Piscataway, N.J.) column. Dextran standards of known molecular weights establish an elution molecular weight profile. Fractions containing labeled material corresponding to a molecular weight of 25 kD, as detected by an appropriate detection method, are collected. The 25 kD β-glucan may be further purified and concentrated by ultracentrifugation with, for example, a Centriprep (Millipore Corp., Bedford, Mass.) with a 50 kD cutoff membrane.

The 25 kD β-glucan prepared by macrophage processing was evaluated against similarly sized β-glucans obtained during the manufacturing process for purifying parent β-glucan (data not shown). The sizes of these β-glucans ranged from about 10 kD to about 30 kD. Interestingly, none of the similarly sized p-glucans tested exhibited the induction activities possessed by the 25 kD β-glucan prepared by macrophage processing. It is evident, therefore, that macrophage processing modifies the 25 kD β-glucan making a unique composition possessing characteristics that are not simply based on size.

### Complement Activating Antibodies

Complement activating antibodies (both naturally found or produced by methods known in the art) are antibodies directed to the tumor or tumor antigens that are able to activate one or more members of the complement cascade. In other words, an antibody that activates complement sufficiently to deposit iC3b on the tumor cells is needed. In certain embodiments, the antibodies are IgG subclass I or IgG subclass II.

The present invention discloses the use of a 25 kD β-glucan with antibodies from essentially any source, including antibodies generated naturally in response to infection, antibodies generated in response to administration of a vaccine, and monoclonal antibodies directly administered as part of a therapy including the use of β-glucan. Any antibody having complement activating features can be used in the methods described herein to enhance β-glucan on tumorcidal activity. The antibody can also be a naturally occurring antibody found in the subject that is able to activate complement sufficiently to allow deposition of iC3b on the tumor cells. Murine antibodies can be raised against any antigen associated with neoplastic (tumor) cells using techniques known in the art. In this regard, tumor cells express increased numbers of various receptors for molecules that can augment their proliferation, many of which are the products of oncogenes. Thus, a number of monoclonal antibodies have been prepared which are directed against receptors for proteins such as transferring, IL-2, and epidermal growth factor. It suffices to say that any antibody that can selectively label antigen—which is to say any antibody—could have its activity enhanced through concurrent administration with β-glucan. This includes antibodies of the various classes, such as IgA, IgD, IgE, and IgM, as well as antibody fragments such as Fab.

The term “antibody” as used herein refers to immunoglobulin molecules and immunologically active portions of immunoglobulin molecules, i.e., molecules that contain an antigen binding site that specifically binds a tumor antigen. A molecule that specifically binds to tumor is a molecule that binds to that polypeptide or a fragment thereof, but does not substantially bind other molecules in a sample, e.g., a biological sample, which naturally contains the polypeptide. Examples of immunologically active portions of immunoglobulin molecules include F(ab) and F(ab′)2 fragments which can be generated by treating the antibody with an enzyme such as pepsin. The term “monoclonal antibody” or “monoclonal antibody composition”, as used herein, refers to a population of antibody molecules that contain only one species of an antigen-binding site capable of immunoreacting with a particular epitope of a target tumor. A monoclonal antibody composition thus typically displays a single binding affinity for a particular polypeptide of the invention with which it immunoreacts.

Polyclonal antibodies can be prepared as described above by immunizing a suitable subject with a desired immunogen, e.g., polypeptide of interest or fragment thereof. The antibody titer in the immunized subject can be monitored over time by standard techniques, such as with an enzyme linked immunosorbent assay (ELISA) using immobilized polypeptide. If desired, the antibody molecules directed against the polypeptide can be isolated from the mammal (e.g., from the blood) and further purified by well-known techniques, such as protein A chromatography to obtain the IgG fraction. At an appropriate time after immunization, e.g., when the antibody titers are highest, antibody-producing cells can be obtained from the subject and used to prepare monoclonal antibodies by standard techniques, such as the hybridoma technique originally described by Kohler and Milstein (1975) Nature, 256: 495-497, the human B cell hybridoma technique (Kozbor, et al. (1983) Immunol. Today, 4: 72), the EBV-hybridoma technique (Cole, et al. (1985), Monoclonal Antibodies and Cancer Therapy, Alan R. Liss, Inc., pp. 77-96) or trioma techniques. The technology for producing hybridomas is well known (see generally Current Protocols in Immunology (1994) Coligan, et al. (eds.) John Wiley & Sons, Inc., New York, N.Y.). Briefly, an immortal cell line (typically a myeloma) is fused to lymphocytes (typically splenocytes) from a mammal immunized with an immunogen as described above, and the culture supernatants of the resulting hybridoma cells are screened to identify a hybridoma producing a monoclonal antibody that binds a polypeptide of interest.

Any of the many well known protocols used for fusing lymphocytes and immortalized cell lines can be applied for the purpose of generating a monoclonal antibody to a polypeptide of interest (see, e.g., Current Protocols in Immunology, supra; Galfre, et al. (1977) Nature, 266: 55052; R. H. Kenneth, in Monoclonal Antibodies: A New Dimension In Biological Analyses, Plenum Publishing Corp., New York, N.Y. (1980); and Lerner (1981) Yale J. Biol. Med., 54: 387-402. Moreover, the ordinarily skilled worker will appreciate that there are many variations of such methods that also would be useful.

Alternative to preparing monoclonal antibody-secreting hybridomas, a monoclonal antibody to a polypeptide of the invention can be identified and isolated by screening a recombinant combinatorial immunoglobulin library (e.g., an antibody phage display library) with the polypeptide to thereby isolate immunoglobulin library members that bind the polypeptide. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 0.240612). Additionally, examples of methods and reagents particularly amenable for use in generating and screening antibody display library can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs, et al. (1991) Bio/Technology, 9: 1370-1372; Hay, et al. (1992) Hum. Antibod. Hybridomas, 3: 81-85; Huse, et al. (1989) Science, 246: 1275-1281; Griffiths, et al. (1993) EMBO J., 12: 725-734.

Additionally, recombinant antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art.

The present invention discloses the use of 25 kD β-glucan with antibodies from essentially any source, including antibodies generated naturally in response to infection, antibodies generated in response to administration of a vaccine, and monoclonal antibodies directly administered as part of a therapy including the use of β-glucan. The majority of humanized mAbs containing the human IgG1 Fc-region have been shown to activate complement, such as Herceptin™ (trastuzumab), Rituxan™ (rituximab), and Erbitux™ (cetuximab) (Spiridon, C. I., et al., Clin. Cancer Res., 8: 1720-1730 (2002), Idusogie, E. E., et al., J. Immunol., 164: 4178-4184 (2000), Cragg, M. S., et al., Blood, 101: 1045-1052 (2003), Herbst, R. S, and Hong, W. K., Semin. Oncol., 29: 18-30 (2002). In certain embodiments the glucan and antibodies work synergistically.

As illustrative of the inventive concept, β-glucans could be locally administered to act synergistically with Herceptin™, a monoclonal antibody sold by Genentech for use in immunotherapy of breast cancer. Herceptin™ is a mAb that recognizes the her2 cell surface antigen which is present on 20% of breast cancer cell types. Clinical trials have demonstrated that Herceptin™ is saving lives, but its effectiveness could be significantly enhanced through concurrent administration of β-glucan. Local administration of glucan along with Herceptin™ therapy could result in a significant increase in the proportion of women responding to Herceptin™ therapy with long lasting remission of their breast cancer. Currently, only 15% of women receiving Herceptin™ therapy show long lasting remission.

Another mAb whose activity is enhanced by whole glucan particles is rituximab, a monoclonal antibody used to treat a type of non-Hodgkin's lymphoma (NHL), a cancer of the immune system. Rituxan™ (rituximab), is effective for patients with low-grade B-cell NHL who have not responded to standard treatments. It targets and destroys white blood cells (B-cells) that have been transformed, resulting in cancerous growth. Rituximab is a genetically engineered version of a mouse antibody that contains both human and mouse components. In the main clinical study of 166 patients with advanced low-grade or slow-growing NHL, which represents about 50% of the 240,000 NHL patients in the United States, tumors shrunk by at least one half in 48% of the patients who completed treatment with rituximab, with 6% having complete remission. B-glucan can be expected to significantly increase the effectiveness of this treatment, by enhancing the destruction of antibody-marked tumor cells.

### Sustained Release

Long-acting formulations of the 25 kD β-glucan can be prepared by stabilizing and encapsulating the glucan into biodegradable microparticle formulations composed of polymers, for example polymers of lactic and glycolic acid.

“Microparticles,” as that term is used herein, includes a biocompatible polymer having the glucan incorporated therein. The biocompatible polymer can include, for example, poly(lactic acid) or a poly(lactic acid-co-glycolic acid) copolymer. The microparticles can be used to deliver the glucan to a patient in need thereof, for example, in a sustained manner. In certain embodiments, the microparticle is delivered locally.

Polymers used in the formulation of the microparticles described herein include any polymer which is biocompatible. Biocompatible polymers suitable for use in the present invention include biodegradable and non-biodegradable polymers and blends and copolymers thereof, as described herein. A polymer is biocompatible if the polymer and any degradation products of the polymer are non-toxic to the patient and also possess no significant deleterious or untoward effects on the patient's body, such as a significant immunological reaction at an injection or implantation site.

“Biodegradable,” as defined herein, means the composition will degrade or erode in vivo to form smaller chemical species. Degradation can result, for example, by enzymatic, chemical and physical processes. Suitable biocompatible, biodegradable polymers include, for example, poly(lactides), poly(glycolides), poly(lactide-co-glycolides), poly(lactic acid)s, poly(glycolic acid)s, polycarbonates, polyesteramides, polyanydrides, poly(amino acids), polyorthoesters, poly(dioxanone)s, poly(alkylene alkylate)s, copolymers or polyethylene glycol and polyorthoester, biodegradable polyurethane, blends thereof, and copolymers thereof.

Suitable biocompatible, non-biodegradable polymers include non-biodegradable polymers such as, for example, polyacrylates, polymers of ethylene-vinyl acetates and other acyl substituted cellulose acetates, non-degradable polyurethanes, polystyrenes, polyvinylchloride, polyvinyl flouride, poly(vinyl imidazole), chlorosulphonate polyolefins, polyethylene oxide, blends thereof, and copolymers thereof.

In certain embodiments, the biocompatible polymer is at least one member selected from the group consisting of poly(lactide)s, poly(glycolide)s, poly(lactide-co-glycolide)s, poly(lactic acid)s, poly(glycolic acid)s, polycarbonates, polyesteramides, polyanhydrides, poly(amino acids), polyorthoesters, polyacetals, polycyanoacrylates, polyetheresters, polycaprolactone, poly(dioxanone)s, poly(alkylene alkylate)s, polyurethanes, and blends and copolymers thereof.

Acceptable molecular weights for biocompatible polymers used in this invention can be determined by a person of ordinary skill in the art taking into consideration factors such as the desired polymer degradation rate, physical properties such as mechanical strength, and the rate of dissolution of polymer in the solvent. Typically, an acceptable range of molecular weight is of about 2,000 Daltons to about 2,000,000 Daltons. In a preferred embodiment, the polymer is a biodegradable polymer or copolymer. In another preferred embodiment, the polymer is a poly(lactide-co-glycolide) (PLG) which can have lactide:glycolide ratios of about 25:75 to about 85:15 such as about 25:75, 50:50, 75:25 and 85:15, and a molecular weight of about 5,000 Daltons to about 150,000 Daltons. In one embodiment, the molecular weight of the PLG has a molecular weight of about 5,000 Daltons to about 42,000 Daltons.

Suitable solvents, e.g., polymer solvents, suitable for production of microparticles can be determined via routine experimentation using techniques well-known to those of ordinary skill in the art. Suitable solvents include, but are not limited to, methylene chloride, acetone, acetic acid, ethyl acetate, methyl acetate, tetrahydrofuran, dimethylsulfoxide (DMSO), methyl ethyl ketone (MEK), acetonitrile, toluene, and chloroform. In one embodiment, the solvent is selected from the group consisting of methylene chloride, chloroform, ethyl acetate, methyl acetate, acetone, acetic acid, acetonitrile, dimethylsulfoxide, methyl ethyl ketone and toluene.

Methods for forming microparticles containing a glucan are described in U.S. Pat. No. 5,019,400, issued to Gombotz, et al., on May 28, 1991; U.S. Pat. No. 5,922,253 issued to Herbert, et al., on Jul. 13, 1999; and U.S. Pat. No. 6,455,074 issued to Tracy, et al., on Sep. 24, 2002, the entire contents of each of which are incorporated herein by reference.

### In Vivo Trafficking of the Soluble β-Glucan

The soluble β-glucan PGG (Biothera, Eagan, Minn.) was labeled with fluoroscein dichlorotriazine (DTAF; Molecular Probes, Eugene, Oreg.) that covalently reacts with hydroxyl groups of polysaccharides using a modification of the procedures suggested by the manufacturer. Groups of five C57BL/6 wild type mice or CR3-deficient mice were given 1200 μg of the DTAF PGG β-glucan by tail vein injection. The spleen and bone marrow from different groups of mice were collected at day 1, day 3, and day 7. Confocal microscopy and flow cytometry were performed to determine the phenotype of fluorescence-positive cells using the following monoclonal antibodies (mAbs) CD3, CD19, Gr-1, F4/80, NK1.1, and CD11c.

### The Isolation of an Active Moiety of Soluble β-Glucans

Murine resident peritoneal macrophages were maintained in a bioreactor flask (antegra Biosciences; Chur, Switzerland) in macrophage growth serum-free medium (SFM medium, Invitrogen, Grand Island, N.Y.). DTAF-labeled β-glucan PGG was added to the culture. Following three weeks of cell culture, the cell-free fluid from the lower chamber of the bioreactor containing soluble fragments of β-glucan PGG was collected. This material was separated by high-performance liquid chromatography (HPLC) (Waters 1525, Waters Corp., Milford, Mass.) utilizing a monophasic gradient and separated on a Sephacryl S-200 (GE Healthcare, formerly Amersham Biosciences, Piscataway, N.J.) column. DTAF-labeled dextran standards of known molecular weights established an elution molecular weight profile. Fractions containing DTAF-labeled material, as detected by A₄₉₀ on a Waters 2996 Photodiode Array Detector, indicated a dominant bimodal peak containing material of 25 kD and a small peak containing remaining parent 150 kD β-glucan PGG. 25 kD fragments were further purified and concentrated by ultracentrifugation with a Centriprep (Millipore Corp., Bedford, Mass.) with a 50 kD cutoff membrane. These fractions were confirmed to contain hexose by the phenol-sulfinuric acid method.

### Collection of Thioglycolate-Elicited Peritoneal Neutrophils in Mice

Three milliliters of BBL® fluid thioglycolate medium (Becton Dickinson, Cockeysville, Md.) were injected intraperitoneally into mice to mobilize the marginated pool of neutrophils from the bone marrow to the peritoneal cavity. Four to six hours following thioglycolate administration, peritoneal-infiltrating neutrophils were collected using a transfer pipette by washing the cavity 4-5 times with 2-3 mL aliquots of ice-cold complete RPMI medium.

### Isolation of Human Peripheral Blood Neutrophils

Utilization of human subjects was approved by the Institutional Review Board (ORB) of the University of Louisville. Following informed consent, peripheral blood was collected and neutrophils were enriched using two densities of Ficoll: 1.077 g/mL and 1.105 g/mL for separation (≧98% neutrophils).

### Direct Binding of 25 kD β-Glucan Fragments and Intact β-Glucan to Neutrophils

Neutrophils were collected from either mice or humans as described above and resuspended in complete RPMI that had been supplemented with 10 μg/mL polymyxin B (Sigma-Aldrich, St. Louis, Mo.) to neutralize adventitious lipopolysaccharide (LPS). Neutrophils and β-glucan were incubated at 37° C. in a 5% CO₂ humidified incubator for 3 hours. Following the incubation, cells were washed three times. Murine neutrophils were incubated with 10 μg/mL Fc block (rat anti-mouse CD16/32 mAb; BDPharmingen, San Diego, Calif.) and human neutrophils were incubated with a 1/20 dilution of heat-inactivated human serum at 0.1 mL total volume for 20 minutes at room temperature to block Fc receptors and to control false-positive staining. In some experiments, murine neutrophils were additionally stained with anti-Gr-1-PE or anti-CD11β-Per CP Cy 5.5. When data were acquired by flow cytometry, the cells were gated by light scatter and propidium iodide exclusion was always utilized on a control aliquot of cells to confirm ≧90% cell viability.

### Induction of CBRM 1/5 Neo-Epitope on Human Neutrophils

Neutrophils were collected from human donors and resuspended in complete RPMI that was supplemented with 10 μg/mL polymyxin B (Sigma-Aldrich, St. Louis, Mo.) to neutralize LPS. As a positive control, an aliquot of neutrophils was not supplemented with polymyxin B and was instead activated with serial dilutions of LPS to induce the neo-epitope. One million neutrophils were added to the wells of 96-well plates and mixed with either the DTAF-labeled parent β-glucan PGG or the DTAF-labeled 25 kD β-glucan active moiety that had been serially diluted in complete RPMI supplemented with polymyxin B or with LPS that had been serially diluted in complete RPMI. Neutrophils and either β-glucan or LPS were incubated at 37° C. in a 5% CO₂ humidified incubator for 3 hours. Following the incubation, the contents of the wells were collected and washed as described above. Inhibition of human Fc receptors was also carried out as described above. The neutrophils were then stained with an optimized dilution of CBRM 1/5 mouse anti-human CD11b mAb that detects the activation neo-epitope of CD11b.

### In Vitro CR3-Dependent Cellular Cytotoxicity

In vitro cytotoxicity of SKOV-3 cells by β-glucan-primed human neutrophils was analyzed using a real-time measure of the impedance of electrical current by viable target cells adhered to a conductor on the bottom of wells in a 96-well plate (Acea Biosciences, Inc., San Diego, Calif.). Briefly, 5×10³ SKOV-3 cells were placed into the wells of the Acea 96-well plates and maintained in McCoys 5A medium. The SKOV-3 cells were allowed to acclimate to the environment within the plate for 24 hours. Following this incubation, fresh human serum was diluted to an equal volume of complete McCoy's medium that contained sufficient trastuzumab to make a 10 μg/mL working dilution and a final volume of 0.1 mL and added to the adherent SKOV-3 cells. The cells were incubated for 30 minutes at 37° C. to permit complement activation and deposition of human iC3b. Human neutrophils, isolated from volunteers as described above, were added to achieve effector-to-target cell ratios of 10:1 and 20:1. Parent β-glucan PGG, or the 25 kD fragments derived from co-culture of macrophages and β-glucan PGG, was added to the neutrophils to prime CR3. The primed neutrophils were added in a final volume of 0.1 mL to the iC3b-opsonized tumor cells. Control wells contained iC3b-opsonized tumors and non-β-glucan-primed neutrophils to measure the contribution of ADCC to the cytotoxicity. Cells were incubated at 37° C. in a humidified 5% CO₂ incubator for 12 hours. The CR3—DCC cytotoxicity of target cells was calculated by measuring the ratio of the cell indices, or the relative decrease in current impedance, among wells containing iC3b-opsonized SKOV-3 cells and β-glucan-primed neutrophils and wells containing iC3b-opsonized SKOV-3 cells and non-β-glucan-primed neutrophils. For some experiments, Syk kinase inhibitor Piceatannal (Sigma-Aldrich, St. Louis, Mo.) or PI 3-kinase inhibitor LY294002 (Calbiochem, Darmstadt, Germany) was added to the cytotoxicity assay.

### Dual Ligation of CR3 and Measurement of Syk Phosphorylation and PI 3-Kinase Activity

Human neutrophils were stimulated with anti-M1/70 mAb (1 μg/ml) followed with goat-anti-rat Ig (5 μg/ml) in the presence or absence of the 25 kD active moiety of β-glucan (10 μg/ml) at 37° C. To detect Syk phosphorylation, cells were stimulated for 30 minutes and lysed by lysis buffer. The supernatants were incubated with a cocktail of anti-CR3 mAbs (OKM-1, MN-41, MO-1, LM-2) and 40 μl of Protein A-agarose for two hours at 4° C. The agarose beads were pelleted, washed three times with lysis buffer, suspended in SDS sample buffer and boiled for five minutes. The immunoprecipitates were analyzed on SDS-PAGE gel. Proteins were transferred onto nitrocellulose membranes and blotted with anti-phospho-Syk antibody or anti-Syk antibody (Santa Cruz Biotechnology, Santa Cruz, Calif.). The bound antibody was detected using enhanced chemiluminescence (Cell Signaling Technology, Beverly, Mass.). For measurement of PI 3-kinase activity, cells were stimulated for one hour and aliquots of cell lysates adjusted for protein concentration (500 μg of protein) were incubated for two hours at 4° C. with anti-PI 3-kinase p85 antibody, and immune complexes were adsorbed onto protein A-agarose for three hours. The complexes were then washed twice with lysis buffer and three times with 10 mM Tris-HCl, pH 7.4. To ensure that PI 3-kinase levels remained equivalent at the end of the immunoprecipitation, 10% from each treatment sample were collected during the last wash in a separate tube and analyzed by SDS-PAGE and immunoblotting with Abs to the p85 PI 3-kinase subunit. PI-3K activity was assayed with PI-3K ELISA kit (Echelon Biosciences, Salt Lake City, Utah) according to manufacturer's instructions (An, H., et al., Src homology 2 domain-containing inositol-5-phosphatase 1 (SHIP1) negatively regulates TLR4-mediated LPS response primarily through a phosphatase activity- and PI-3K-independent mechanism, Blood 105, 4685-92 (2005)). In brief, cell lysates were prepared and PI-3K was immunoprecipitated with antibody against the p85 subunit and incubated with PI(4,5)P2 (phosphatidylinositol-4,5-diphosphate). The reaction products were incubated with a PI(3,4,5)P3 detector protein then added to a PI(3,4,5)P3-coated microplate for competitive binding. A peroxidase-linked secondary detection reagent and colorimetric detection were used to detect PI(3,4,5)P3 detector protein binding to the plate. The colorimetric signal was inversely proportional to the amount of PI(3,4,5)P3 produced by PI-3K activity.

### In Vivo Tumor Therapy

RMA-S-MUC1 is a C57BL/6 T cell lymphoma that does not express MHC class I as a consequence of a mutation in the gene encoding the transporter associated with Ag presentation (TAP) protein, and was kindly provided by Dr. Olivera J. Finn (University of Pittsburgh). Briefly, 1×10⁶ RMA-S-MUC1 cells were implanted subcutaneously in a mammary fat pad of wild-type C57BL/6 or CR3-deficient mice (n=10). After seven days, when tumors of 1-2 mm appeared, the mice were divided into groups of ten and therapy was initiated with 14 G2a antiGD2 mAb or BCP8 anti-MUC1 mAb (200 μg intravenously (i.v.) twice weekly) with or without i.v. β-glucan PGG (1200 μg/mouse, twice a week). Therapy was continued for three weeks during which time tumor measurements by calipers were calculated as the average of perpendicular diameters twice weekly. Mice were sacrificed when tumors reached 12 mm in diameter. Survival was monitored for a period of 100 days beyond the tumor implantation.

### Statistical Analysis

Data were entered into Prism 4.0 (Graph Pad Software, San Diego, Calif.) to generate graphs of percentage of fluorescent positive cells or tumor regression, and Student's ‘t’ test was employed to determine the significance of differences between two data sets. Survival curves were created using the Kaplan-Meier method and statistical analyses of survival curves utilized a log-rank test.

The above materials and methods were used in the experiments below:

### EXPERIMENT 1

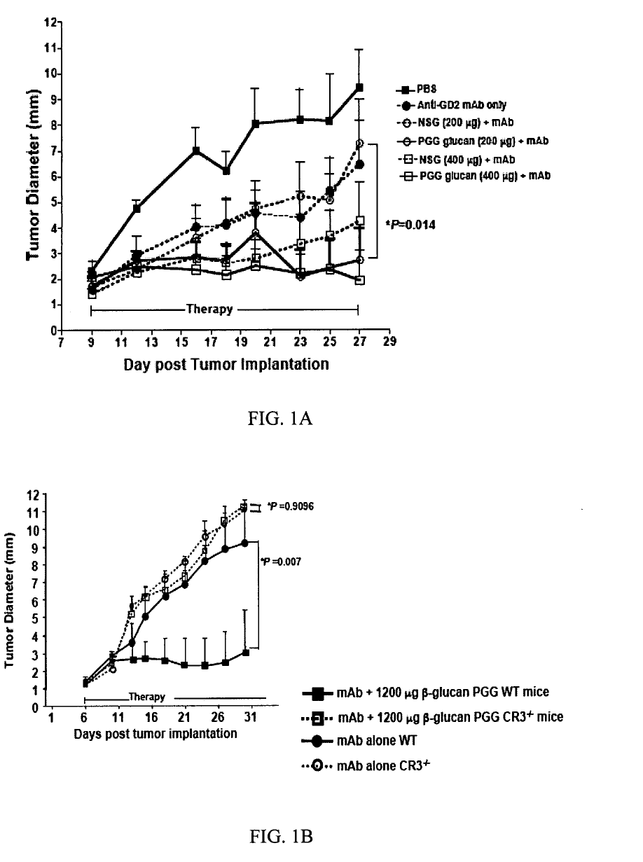

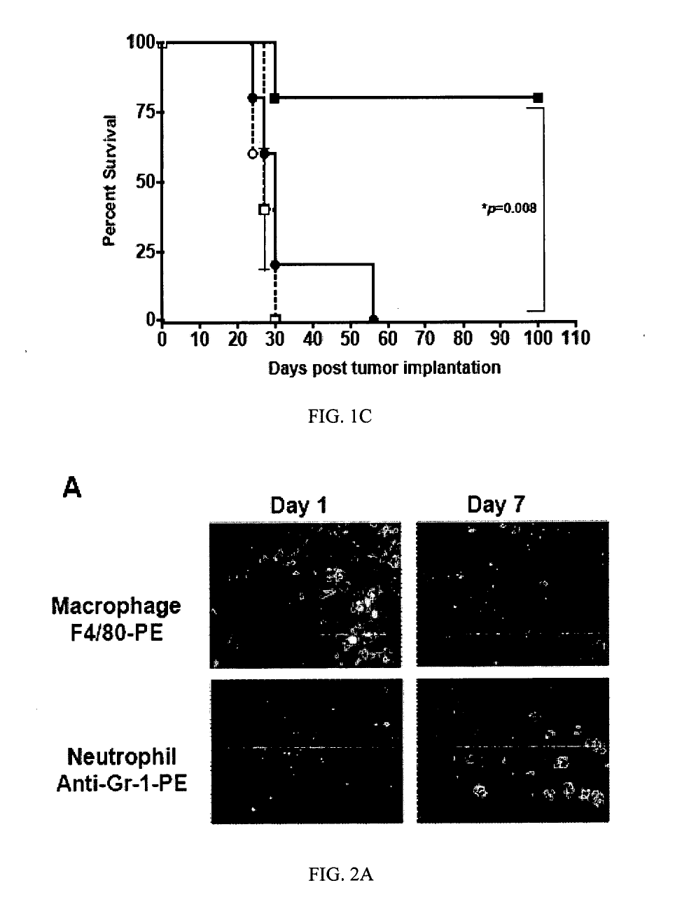

C57B1/6 mice or CR3-deficient mice (n=10) were implanted subcutaneously with RMA-S-MUC1 cells, and a tumor was allowed to form over seven days before initiating immunotherapy. Mice were treated with BCP-8 anti-MUC1 mAb (200 μg twice a week) and/or β-glucan PGG (1200 μg twice a week) for a total of three weeks. Tumor growth (FIG. 1a-1b) and survival (FIG. 1c) were monitored. Mean values±SE of the mean are shown.

### Results:

Intravenous administration of β-glucan PGG twice a week to tumor-bearing mice, in combination with an anti-tumor mAb, almost completely suppressed tumor growth and greatly increased survival, whereas administration of mAb alone had no effect (FIG. 1a,b). In contrast, tumor-bearing mice treated with nothing (PBS) (not shown), β-glucan alone (not shown) or mAb alone had similar unrestrained tumor growth (FIG. 1a). This tumor suppressive effect of β-glucan with mAb was not seen in CR3-deficient (CR3^{-/-}) mice, suggesting that soluble β-glucan mediated tumor therapy is CR3 dependent.

### EXPERIMENT 2

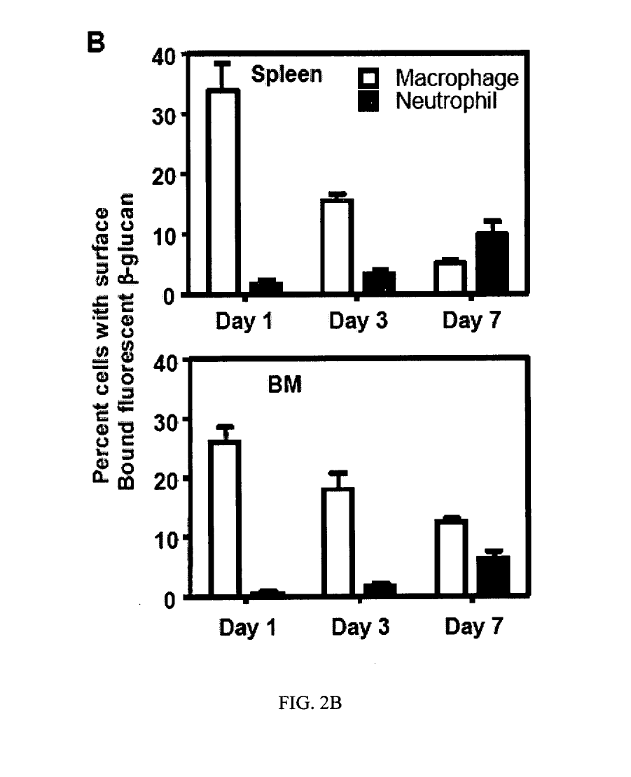

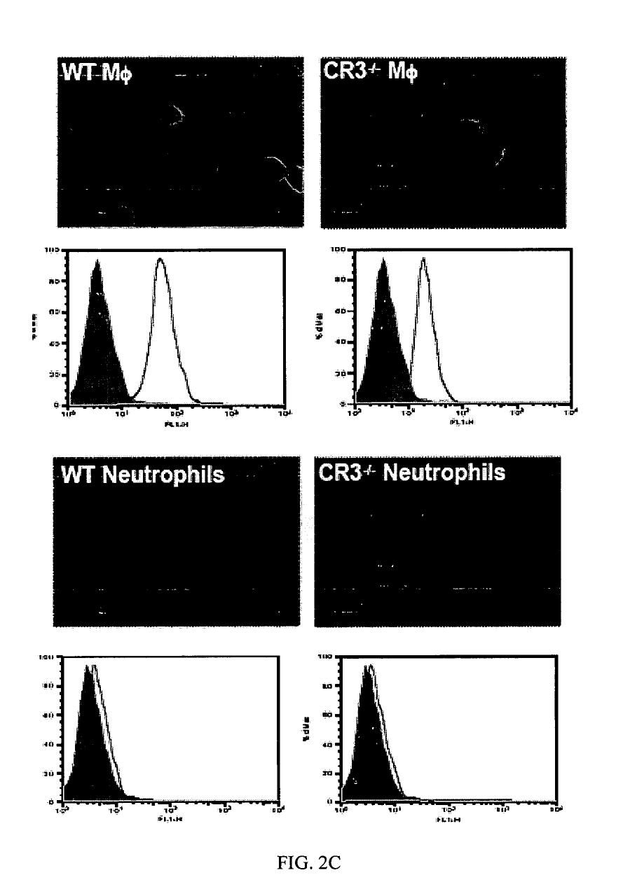

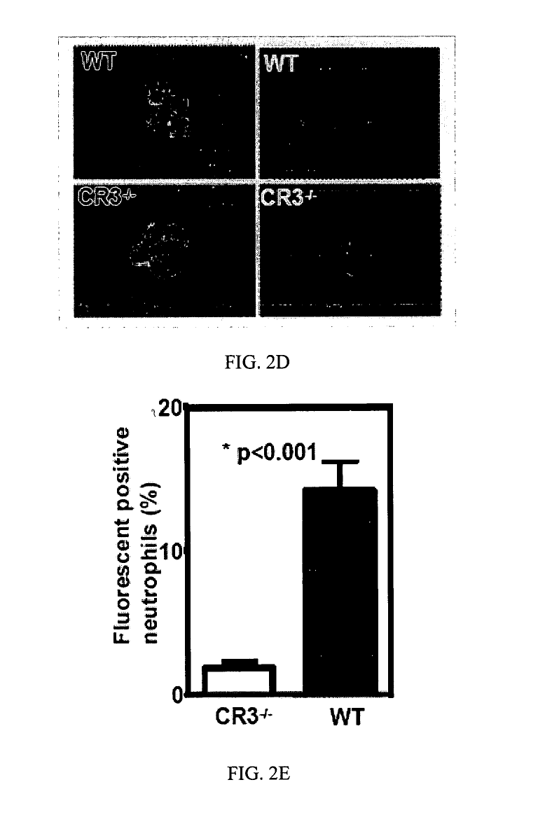

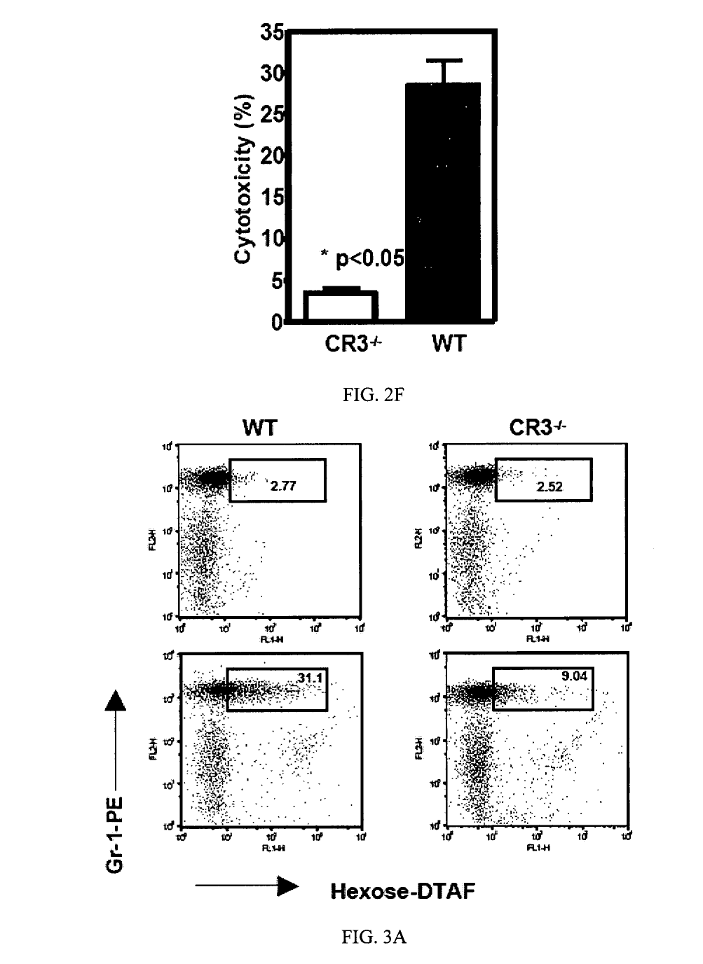

Mice were given fluorescein DATF-labeled PGG β-glucan (green) intravenously and were sacrificed at day 1, day 3, and day 7. The spleens were frozen-sectioned and slides were stained with F4/80-PE or anti-Gr-1-PE (red). Original magnification was 60× (FIG. 2a,b). Cells from the spleen or bone marrow (BM) were stained with F4/80, anti-Gr-1 mAbs and analyzed by flow cytometry. The cells with surface bound fluorescent β-glucan were gated on F4/80⁺ or Gr-1⁺ cells (n=5). Thioglycolate elicited macrophages or neutrophils from wildtype (WT) or CR3-deficient (CR3^{-/-}) mice were incubated with DTAF-labeled PGG β-glucan (green). Cells were observed under confocal microscopy and also analyzed by flow cytometry, respectively. Original magnification was 100× (FIG. 2c). Peritoneal neutrophils from WT and CR3^{-/-} mice receiving DTAF-labeled β-glucan PGG were marginated by thioglycolate injection at day 7 and stained with anti-Gr-1-PE (red). Original magnification was 100× (FIG. 2d). Neutrophils from above were then stained with anti-Gr-1 mAb and analyzed by flow cytometry. The cells with surface bound fluorescent β-glucan were gated on Gr-1⁺ cells (n=5) (FIG. 2e). The neutrophils from above were also assayed for cytotoxicity using iC3b-opsonized RMA-S-MUC1 tumor cells as targets as described in the methods (FIG. 2f).

### Results:

One day following administration of fluorescein-labeled β-glucan to mice, the polysaccharide appeared in splenic Mφ, but not in neutrophils (FIG. 2a). However, seven days following injection, β-glucan positive macrophages virtually disappeared from the spleen while approximately 10% of neutrophils, in both the spleen and bone marrow, were now found to contain β-glucan (FIG. 2a,b).

Thioglycolate-elicited peritoneal Mφ and neutrophils from wildtype (WT) and CR3^{-/-} mice were harvested and incubated with fluorescein-labeled β-glucan to determine the possible importance of CR3. Macrophages from WT and CR3^{-/-} mice had comparable uptake of α-glucan as assessed by both fluorescence microscopy and FACS analysis, suggesting that the uptake of intact β-glucan by Mφ is CR3-independent (FIG. 2c). The uptake of labeled β-glucan was blocked by a 10-fold excess of unlabeled β-glucan (data not shown). Interestingly, the labeled β-glucan did not bind to the neutrophils in these preparations, suggesting that the β-glucan that bound to neutrophils in vivo seven days following administration (FIG. 2a) might be a modified form of the parent β-glucan, perhaps arising from processing of the original material by Mφ and subsequent release of the β-glucan fragments to neutrophils. Peritoneal neutrophils, elicited by thioglycolate, were then examined from WT and CR3^{-/-} mice seven days after administration of labeled β-glucan to further explore the importance of CR3 in the priming of neutrophils per se. Granulocytes from WT mice that had not been given α-glucan served as a control for the ability of non-glucan-exposed neutrophils to kill iC3b-coated tumor cells. Neutrophils from WT mice, but not CR3^{-/-} mice exhibited β-glucan binding by both fluorescence microscopy (FIG. 2d) and FACS analysis (FIG. 2e). Furthermore, these WT neutrophils with surface-bound β-glucan were capable of killing iC3b-opsonized RMA-S-MUC1 tumor cells. The requirement for CR3 was confirmed by abolished tumor killing mediated by neutrophils from CR3^{-/-} mice (FIG. 2f). Therefore, uptake of parent β-glucan by Mφ does not require CR3. However, degradation fragments, presumably the active fragments, released from Mφ are bound by WT but not CR3^{-/-} neutrophils, and the former are capable of killing iC3b-opsonized tumor cells.

### EXPERIMENT 3

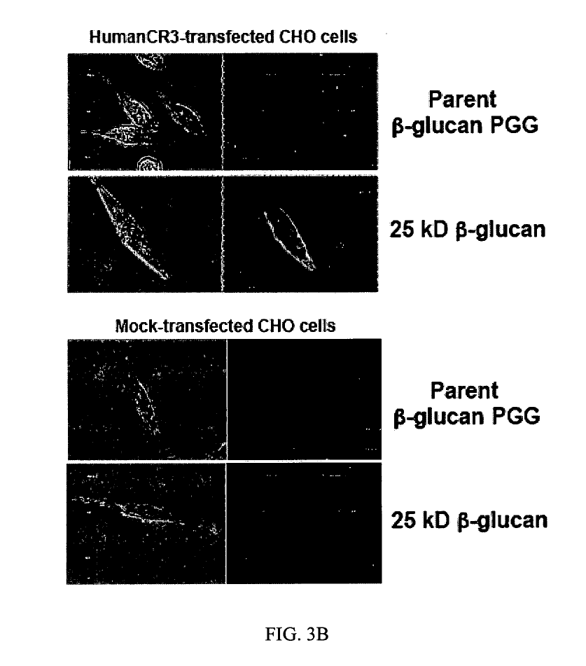

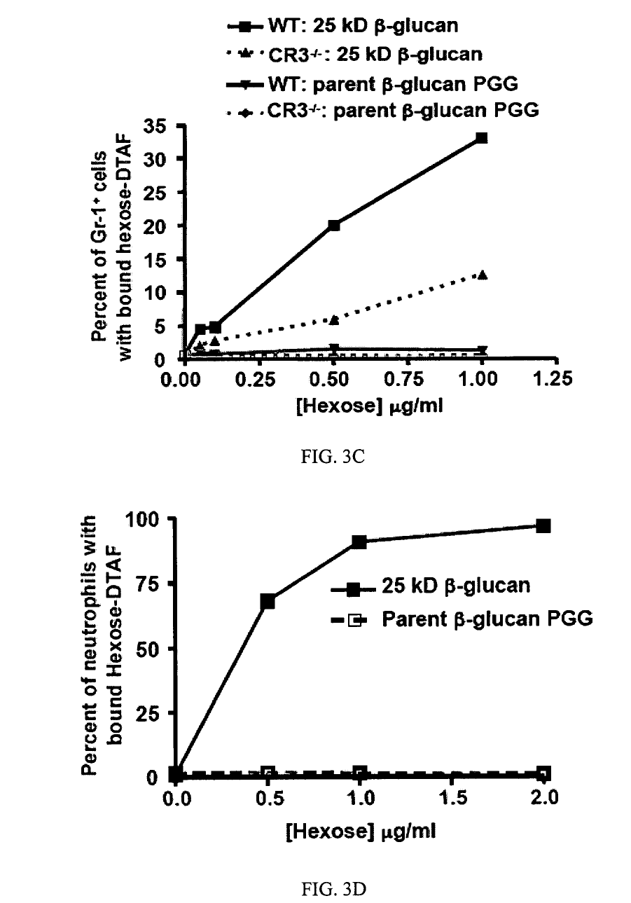

Peritoneal neutrophils from WT and CR3^{-/-} mice were stained with DTAF-labeled β-glucan PGG (FIG. 3a-upper panel) or with DTAF-labeled 25 kD active moiety released from macrophage culture (FIG. 3a-lower panel). Human CR3 transfected CHO cells were cultured in glass-plates and stained with anti-CR3-PE (red) and DTAF-labeled parent β-glucan PGG or 25 kD β-glucan (green) (FIG. 3b). Slides were observed under Nikon fluorescent microscope. Original magnification was 20×. Peritoneal neutrophils from WT and CR3^{-/-} mice or human peripheral blood neutrophils were stained with various amounts of DTAF-labeled parent β-glucan PGG or 25 kD β-glucan. The cells with surface bound fluorescent β-glucan were gated on Gr-1⁺ cells (FIG. 3c,d). Human peripheral blood neutrophils were incubated with varying concentrations of parent β-glucan PGG or 25 kD β-glucan and stained with anti-CBRM1/5 mAb. Human neutrophils were co-cultured with iC3b-opsonized SKOV-3 tumor cells in the presence of varying concentrations of parent β-glucan PGG or 25 kD β-glucan for cytotoxicity assay as described in the methods. The E:T ratio was 20:1.

### Results:

In an attempt to identify the nature of this putative active fragment, an in vitro Mφ culture system was used in which resident peritoneal Mφ were exposed to fluoroscein-labeled intact β-glucan. Long-term incubation resulted in the appearance of a β-glucan fragment with an approximate molecular size of 25 kD by high-resolution high-performance liquid chromatography. This fragment, but not the parent β-glucan, bound directly to mouse and human neutrophils or CR3 transfected CHO cells (FIG. 3a,b). The binding of this 25 kD β-glucan fragment by mouse neutrophils was CR3-dependent (FIG. 3c) and saturable (FIG. 3d). Thus, it appears that the 25 kD β-glucan fragment, but not the 150 kD parent molecule, mediates CR3-dependent binding to neutrophils and subsequent biological functions.

Human neutrophils were exposed to this fragment to further characterize the bioactivity of the 25 kD β-glucan and the appearance of the “activated” epitope of CD11b/CD18 (CR3), detected with the mAb CBRM 1/5, was assessed. Neutrophils stimulated by the 25 kD β-glucan fragment, but not parent β-glucan, induced CBRM 1/5 expression in a dose-dependent manner. In these experiments, exogenous LPS contamination was controlled by maintaining all buffers with 10 μg/ml polymyxin B. These data indicate that the 25 kD α-glucan fragment could effect the CR3-dependent priming of neutrophils and might promote neutrophil killing of iC3b-opsonized targets. Indeed, the 25 kD β-glucan, but not parent β-glucan, greatly amplified the cytotoxicity of neutrophils against iC3β-opsonized human ovarian carcinoma cells in a dose-dependent fashion. Therefore, the 25 kD β-glucan, not the parent β-glucan, is necessary and sufficient for CR3-dependent, neutrophil-mediated cytotoxicity against iC3b-opsonized tumor cells.

### EXPERIMENT 4

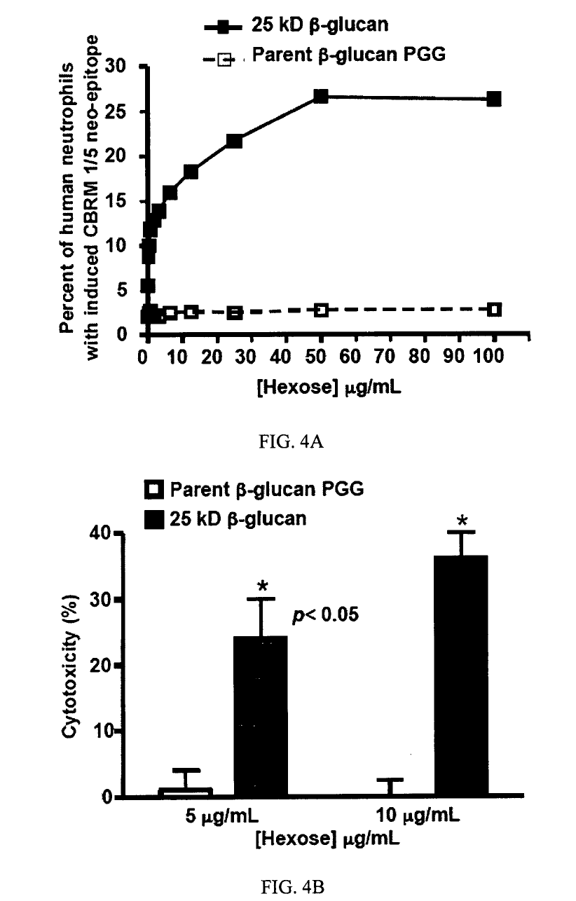

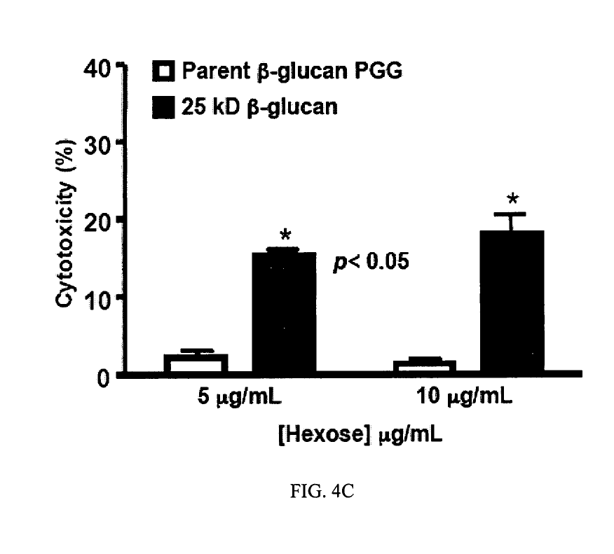

Human peripheral blood neutrophils were stimulated with rat anti-human/mouse CR3 I-domain mAb M1/70 followed by goat anti-rat secondary antibody (with or without 25 kD β-glucan) or 25 kD β-glucan alone for 30 minutes. Cell lysates were immunoprecipitated with a cocktail of anti-CR3 mAbs and the immunoprecipitates were analyzed on SDS-PAGE gel. Proteins were transferred onto nitrocellulose membranes and blotted with anti-Syk antibody or anti-phospho-Syk antibody, respectively (FIG. 4a).

Human peripheral blood neutrophils were also stimulated with 25 kD β-glucan, M1/70 mAb followed by secondary Ab, or both for 30 minutes. Cells were fixed, permeabilized and stained with antiphospho-Syk mAb or isotype control antibody. Cells were assessed by flow cytometry. Mean fluorescence intensity was compared in each stimulation condition (FIG. 4b).

Additionally, human peripheral blood neutrophils were stimulated with M1/70 mAb followed by secondary Ab (with or without 25 kD β-glucan) or 25 kD β-glucan alone for one hour. Cell lysates were immunoprecipitated with anti-PI 3-kinase p85 mAb. The immunoprecipitates were analyzed on SDS-PAGE gel and blotted with anti-PI 3-kinase p85 mAb. The immunoprecipitates were also measured for PI 3-kinase activity by ELISA. The PI 3-kinase activity was represented as the level of PI(3,4,5)P3 (PI3P3) (FIG. 4c).

Human peripheral blood neutrophils were also stimulated with M1/70 mAb followed by secondary Ab with 25 kD β-glucan in the presence or absence of PI 3-kinase inhibitor LY294002 (50 μM) and/or Syk kinase inhibitor Piceatannol (25 μM) for one hour. Cells were immunoprecipitated with anti-PI 3-kinase p85 mAb and PI 3-kinase activity was measured by ELISA. The PI 3-kinase activity was arbitrarily setup as 100% for neutrophils stimulated with dual ligation (31.45±3.8 μM). The percentage of PI 3-kinase activity was generated by PI 3-kinase activity from inhibitor treated cells divided by that from dual ligation stimulated cells (9.05±1.14 μM for LY294002 and 17.1±1.5 μM for Piceatannol, respectively).

In addition, human neutrophils were co-cultured with iC3b-opsonized SKOV-3 tumor cells and 25 kD β-glucan in the presence or absence of PI 3-kinase inhibitor LY294002 (50 μM) and/or Syk kinase inhibitor Piceatannol (25 μM). The E:T ratio was 20:1. The cytotoxicity was arbitrarily set up as 100% for neutrophils stimulated with 25 kD β-glucan (35.7%±3.61%). The percentage of CR3-dependent cellular cytotoxicity was generated by cytotoxicity from the inhibitor treated group divided by that from the non-treated group (7.88%±1.39% for LY294002 and 16.150%±2.68% for Piceatannol, respectively).

### Results:

Syk phosphorylation was enhanced by dual ligation of CR3 and co-precipitated with CR3. This was confirmed by western blot (FIG. 4a) and intracellular anti-phospho-Syk Ab staining assessed by flow cytometry (FIG. 4b). The results indicate that CR3-dependent cytotoxicity against iC3b-opsonized yeast or tumor cells requires simultaneous ligation of two distinct binding sites in CR3: one for iC3b and the second for β-glucan (Vetvicka, V., Thornton, B. P. & Ross, G. D., “Soluble β-glucan polysaccharide binding to the lectin site of neutrophil or natural killer cell complement receptor type 3 (CD11b/CD18) generates a primed state of the receptor capable of mediating cytotoxicity of iC3b-opsonized target cells,” J. Clin. Invest. 98, 50-61 (1996); Vetvicka, V., Thornton, B. P., Wieman, T. J. & Ross, G. D., “Targeting of natural killer cells to mammary carcinoma via naturally occurring tumor cell-bound iC3b and β-glucanprimed CR3 (CD11b/CD18),” J. Immunol. 159, 599-605 (1997)). However, it remains unknown how this dual ligation acts to prompt neutrophil cytotoxicity. Prior investigations of integrin signaling in phagocytes demonstrated a hierarchical activation of the Src-family kinase and Syk (Berton, G., et al., Src and Syk kinases: key regulators of phagocytic cell activation. Trends Immunol 26, 208-14 (2005)). Syk activation in particular may be crucially important because immobilized anti-CD11b induces a respiratory burst in WT but not Syk-deficient neutrophils (Mocsai, A., et al., Syk is required for integrin signaling in neutrophils, Immunity 16, 547-58 (2002)).

Following CR3 dual ligation, increased PI 3-kinase activity compared to Ab or glucan stimulation only was observed (FIG. 4c). In further support of the activation of PI 3-kinase by phospho-Syk, it was observed that the Syk kinase inhibitor, Piceatannal (25 μM), significantly blocked this increase in PI 3-kinase activity. This particular mechanism of signaling appears to be crucial because both the PI 3-kinase inhibitor, LY294002 (50 μM), and the Syk kinase inhibitor, Piceatannal (25 μM), potently blocked dual ligation-mediated cytotoxicity. Abrogation of cytotoxicity was proportional to the inhibition of PI 3-kinase activity mediated by either inhibitor and a higher dose of the PI 3-kinase inhibitor, LY294002 (100 μM), completely abrogated β-glucan mediated cytotoxicity (data not shown).

The work presented here provides a more complete picture of the mechanisms involved in the anti-tumor effects of β-glucan in combination with anti-tumor mAbs. Intact β-glucan is first taken up by Mφ and cleaved into a 25 kD active fragment. This active fragment binds to neutrophil CR3 and primes these cells for target killing through signaling events involving both Syk and PI 3-kinase. These data provide a rationale for combining yeast β-glucan with complement activating anti-tumor mAbs such as Rituxan®, (rituximab, Biogen Idec, Mass.) and Herceptin®, (trastuzumab, Genetech, Inc., CA) to promote CR3-dependent attack on tumors.

The accompanying research article entitled “Yeast β-Glucan Amplifies Phagocyte Killing of iC3b-opsonized Tumor Cells via CR3-Syk-PI3-kinase Pathway” by Li et al. is hereby incorporated by reference.

While this invention has been particularly shown and described with references to preferred embodiments thereof, it will be understood by those skilled in the art that various changes in form and details may be made therein without departing from the scope of the invention encompassed by the appended claims.

What is claimed is:

1. A local delivery composition comprising a 25 kD β-glucan with 1,3 linked glucose with 1,6 glucose branches with 1,3 linked glucose side chains for local administration to a tumor site.

2. The composition of claim 1, further comprising a monoclonal antibody.

3. The composition of claim 1, wherein the composition is a sustained release composition.

4. A method of treating tumors comprising locally administering to the tumor the composition of claim 1 and a monoclonal antibody.

5. A method of enhancing the CR3-dependent neutrophil-mediated cytotoxicity against iC3β-opsonized tumor cells, comprising locally administering a 25 kD size β-glucan to the tumor site and a monoclonal antibody, wherein the CR3-dependent neutrophil-mediated cytotoxicity against iC3b-opsonized tumor cells is enhanced.

6. The method of claim 4, wherein intracellular signaling via Syk phosphorylation is enhanced.
